(19) United States
(12) Patent Application Publication
(10) Pub. No.: US 2010/0094407 A1
(43) Pub. Date: Apr. 15, 2010

(54) Multiple Bioactive Agent Eluting Stents

(75) Inventors: Natividad Vasquez, Windsor, CA (US); Iskender Matt Bilge, San Francisco, CA (US); Susan Rea Peterson, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/249,594

(22) Filed: Oct. 10, 2008

(51) Int. Cl. A61L 27/54; A61F 2/82

(52) U.S. Cl. 623/1.42; 427/2.25

(57) ABSTRACT

The apparatus and methods of the present invention in a broad aspect provide novel multiple bioactive agent eluting stents for treating vascular diseases and conditions. Controlled elution of bioactive agents is achieved by the presence of the bioactive agents themselves. One or more characteristics of the bioactive agents cause variations in elution rates or profiles or the other bioactive agents.

### FIELD OF THE INVENTION

The present disclosure relates to apparatus and methods for multiple bioactive agent controlled elution useful for treating, for example, vascular diseases and conditions.

### BACKGROUND OF THE INVENTION

Stents are generally cylindrical shaped devices which are radially expandable to hold open a segment of a vessel or other anatomical lumen after implantation into the body lumen. Stents have been developed with coatings to deliver bioactive agents or other therapeutic agents. Various types of stents are in use, including expandable and self-expanding stents. Expandable stents generally are conveyed to the area to be treated on balloon catheters or other expandable devices. For insertion, the stent is positioned in a compressed configuration along the delivery device, for example, crimped onto a balloon that is folded or otherwise wrapped about a guide wire that is part of the delivery device. After the stent is positioned across the lesion, it is expanded by the delivery device, causing the length of the stent to contract and the diameter to expand. For a self-expanding stent, commonly a sheath is retracted, allowing expansion of the stent.

Stents are used in conjunction with balloon catheters in a variety of medical therapeutic applications including intravascular angioplasty. For example, a balloon catheter device is inflated during PTCA (percutaneous transluminal coronary angioplasty) to dilate a stenotic blood vessel. The stenosis may be the result of a lesion such as a plaque or thrombus. After inflation, the pressurized balloon exerts a compressive force on the lesion, thereby increasing the inner diameter of the affected vessel. The increased interior vessel diameter facilitates improved blood flow. Soon after the procedure, however, a significant proportion of treated vessels re-narrow.

Restenosis associated with interventional procedures such as balloon angioplasty may occur by two mechanisms: thrombosis and intimal hyperplasia. During angioplasty, a balloon is inflated within an affected vessel thereby compressing the blockage and imparting a significant force, and subsequent trauma, upon the vessel wall. The natural antithrombogenic lining of the vessel lumen may become damaged thereby exposing thrombogenic cellular components, such as matrix proteins. The cellular components, along with the generally antithrombogenic nature of any implanted materials (e.g., a stent), may lead to the formation of a thrombus, or blood clot. The risk of thrombosis is generally greatest immediately after the angioplasty.

The second mechanism of restenosis is intimal hyperplasia, or excessive tissue re-growth. The trauma imparted upon the vessel wall from the angioplasty is generally believed to be an important factor contributing to hyperplasia. This exuberant cellular growth may lead to vessel “scarring” and significant restenosis. The risk of hyperplasia associated restenosis is usually greatest 3 to 6 months after the procedure.

Prosthetic devices, such as stents or grafts, may be implanted during interventional procedures such as balloon angioplasty to reduce the incidence of vessel restenosis. To improve device effectiveness, stents may be coated with one or more therapeutic agents providing a mode of localized bioactive agent delivery. The therapeutic agents are typically intended to limit or prevent the aforementioned mechanisms of restenosis. For example, antithrombogenic agents such as heparin or clotting cascade IIb/IIIa inhibitors (e.g., abciximab and eptifibatide) may be coated on the stent thereby diminishing thrombus formation. Such agents may effectively limit clot formation at or near the implanted device. Some antithrombogenic agents, however, may not be effective against intimal hyperplasia. Therefore, the stent may also be coated with antiproliferative agents or other compounds to reduce excessive endothelial re-growth. Therapeutic agents provided as coatings on implantable medical devices may effectively limit restenosis and reduce the need for repeated treatments.

Stents can be coated with a polymer or combination of a polymer and a pharmaceutical agent or bioactive agent. In many of the current medical devices or stent coating methods, a composition of a bioactive agent and a polymer in a solvent is applied to a device to form a substantially uniform layer of bioactive agent and polymer. A common solvent for the polymers and bioactive agents employed is usually required, and techniques have been developed to micronize the bioactive agents into small particles so that the bioactive agents can be suspended in the polymer solution.

Bioactive agents with different mechanisms of action possibly could be used in combination to take advantage of specific properties (such as anti-restenotic properties) of each compound. Further, it is desirable to have controlled elution of bioactive agents from stents. Depending on the type of bioactive agent which is on a stent it may be desirable to have a faster or slower elution rate. For example, when the vessel wall cannot easily absorb a bioactive agent it would not be desirable to have a fast elution rate because the eluted bioactive agent will not be incorporated into the vessel wall. However, this may cause unwanted systemic dissemination of a bioactive agent which may be an inherently toxic substance such as paclitaxel. When the bioactive agent is easily absorbable by the vessel wall, it may be desirable to have quicker elution so that there is less of a chance for systemic dissemination of the bioactive agent.

Various methods have been used to control elution of bioactive agents from stents. One of these methods has been the use of polymers for coating stent with a bioactive agent-polymer mixture. Various parameters may be changed with regards to the polymers to achieve controlled elution.

It would be desirable to find other ways of controlling the elution rates of bioactive agents from stents which do not use the methods discussed above.

### SUMMARY OF THE INVENTION

It is an object of the present disclosure to control the elution of bioactive agents from apparatus such as stents. Such control may be achieved surprisingly by the presence of the bioactive agents themselves. The characteristics of the bioactive agents may affect their relative elution rates or profiles. Therefore, when it is desirable to have two or more bioactive agents which need to elute from a stent, particular bioactive agents may be chosen to achieve a particular elution rate or profile. Further, the quantity of the different bioactive agents may be varied resulting in different elution rates.

In one embodiment, the present disclosure relates to a multiple bioactive agent eluting stent comprising a stent framework, at least two bioactive agents on the stent framework, wherein one or more of the at least two bioactive agents affects the elution rate of one or more of the other of the at least two bioactive agents.

Alternatively, the multiple bioactive agent eluting stent comprises a stent framework, two bioactive agents on the stent framework, wherein one bioactive agent affects the elution rate of the other bioactive agent.

In another embodiment, the one or more of the at least two bioactive agents increases the elution rate of one or more of the other of the at least two bioactive agents. Alternatively, the one or more of the at least two bioactive agents decreases the elution rate of one or more of the other of the at least two bioactive agents.

In another embodiment, the effect on elution rate is caused by at least one characteristic of one or more of the at least two bioactive agents. This characteristic may be, for example, hydrophobicity, diffusivity or molecular size.

In another embodiment, the stent framework comprises a metallic base or a polymeric base. Alternatively, the stent framework comprises a material selected from the group consisting of stainless steel, nitinol, tantalum, MP35N alloy, platinum, titanium, a biocompatible alloy, a biocompatible polymer and a combination thereof.

In another embodiment, the multiple bioactive agent eluting stent further comprises one or more polymers on said stent framework.

In another embodiment, the at least two bioactive agents are located in two or more layers on the stent framework. Alternatively, each of the at least two bioactive agents is located in separate layers on the stent framework.

In another embodiment, all of the at least two bioactive agents are in one layer on the stent framework.

In another embodiment, for the multiple bioactive agent eluting stent of the present disclosure, one or more bioactive agents in an inner layer affects the elution rate of one or more bioactive agents in an outer layer.

In another embodiment, at least one of the bioactive agents is selected from the group consisting of an antiproliferative agent, an anti-inflammatory agent, FKBP-12 binding compound, an antisense agent, an antineoplastic agent, an antibiotic, a therapeutic peptide, a gene therapy agent, a therapeutic substance, an organic bioactive agent, a pharmaceutical compound, a recombinant DNA product, a recombinant RNA product, a collagen, a collagenic derivative, a protein, a protein analog, a saccharide, a saccharide derivative, and a combination thereof. Alternatively, the at least one of the bioactive agents is an antiproliferative agent and at least one of the bioactive agents is an anti-inflammatory agent. Alternatively, the antiproliferative agent is selected from the group consisting of zotarolimus, sirolimus, tacrolimus, everolimus, temsirolimus, and the anti-inflammatory agent is selected from the group consisting of glucocorticoids, cyclooxygenase inhibitors, and fluocinolone Alternatively, the antiproliferative agent is zotarolimus (also referred to herein as A24) and the anti-inflammatory agent is fluocinolone (also referred to herein as A109).

In another embodiment, the one or more polymers is/are selected from the group consisting of urethanes, polylactides, poly-l-lactic acids, polyglycolic acids, polycaprolactones, polyacrylates, polymethacrylates, polymethylmethacrylates, and combinations and/or copolymers thereof.

In another embodiment, the one or more polymers on said stent framework are 60 wt % Butyl methacrylate, 40 wt % Vinyl acetate random copolymer (C10), 48 wt % Hexyl methacrylate, 27 wt % 1-Vinyl-2-pyrrolidinone, 25 wt % Vinyl acetate random terpolymer (C19), and polyvinylpyrrolidone (PVP). Alternatively, the ratio of C10:C19:PVP can be for example, about 30-50:40-70:1-20 or about 38:57:5.

The present disclosure also relates to a method of making a multiple bioactive agent eluting stent comprising the steps of providing a stent framework, applying at least two bioactive agents onto the stent framework, wherein one or more of the at least two bioactive agents affects the elution rate of one or more of the other of the at least two bioactive agents.

In another embodiment, the method of making a bioactive agent eluting stent comprises the steps of providing a stent framework, applying at least two bioactive agents and one or more polymers onto the stent framework, wherein one or more of the at least two bioactive agents affects the elution rate of one or more of the other of the at least two bioactive agents.

In another embodiment, for the presently disclosed methods, the effect on elution rate is caused by at least one characteristic of one more of the at least two bioactive agents. This characteristic may be, for example, hydrophobicity, diffusivity or molecular size.

In another embodiment, for the presently disclosed methods, each of the at least two bioactive agents is located in a separate layer on the stent framework. In another embodiment, for the presently disclosed methods, all of the at least two bioactive agents are in one layer on the stent framework.

In another embodiment, for the presently disclosed methods, the stent framework comprises a material selected from the group consisting of stainless steel, nitinol, tantalum, MP35N alloy, platinum, titanium, a biocompatible alloy, a biocompatible polymer and a combination thereof.

In another embodiment, for the presently disclosed methods, at least one of the bioactive agents is selected from the group consisting of an antiproliferative agent, an anti-inflammatory agent, FKBP-12 binding compound, an antisense agent, an antineoplastic agent, an antibiotic, a therapeutic peptide, a gene therapy agent, a therapeutic substance, an organic bioactive agent, a pharmaceutical compound, a recombinant DNA product, a recombinant RNA product, a collagen, a collagenic derivative, a protein, a protein analog, a saccharide, a saccharide derivative, and a combination thereof. Alternatively, at least one of the bioactive agents is an antiproliferative agent and at least one of the bioactive agents is an anti-inflammatory agent. Alternatively, the antiproliferative agent is selected from the group consisting of zotarolimus, sirolimus, tacrolimus, everolimus, temsirolimus, and the anti-inflammatory agent is selected from the group consisting of glucocorticoids, cyclooxygenase inhibitors, and fluocinolone. Alternatively, the antiproliferative agent is zotarolimus (also referred to herein as A24) and the anti-inflammatory agent is fluocinolone (also referred to herein as A109).

In another embodiment, for the presently disclosed methods, the bioactive agent eluting stent further comprises one or more polymers located on the stent framework. These polymers may be for example, urethanes, polylactides, poly-l-lactic acids, polyglycolic acids, polycaprolactones, polyacrylates, polymethacrylates, polymethylmethacrylates, and combinations and/or copolymers thereof. Alternatively, these polymers may be for example C10, C19, or PVP. Also, the ratio of C10:C19:PVP can be for example, about 30-50:40-70:1-20 or about 38:57:5.

### BRIEF DESCRIPTION OF THE DRAWINGS

FIG. 1 shows the cumulative elution of fluocinolone (A109) (μg) compared to the fluocinolone (A109)/zotarolimus (A24) combination.

FIG. 2 shows the daily elution of fluocinolone (A109) (μg) compared to the fluocinolone (A109)/zotarolimus (A24) combination.

FIG. 3 shows the cumulative elution of fluocinolone (A109) (percent) compared to the fluocinolone (A109)/zotarolimus (A24) combination.

FIG. 4 shows the cumulative elution of zotarolimus (μg) compared to the fluocinolone (A109)/zotarolimus (A24) combination.

FIG. 5 shows the daily elution of zotarolimus (μg) compared to the fluocinolone (A109)/zotarolimus (A24) combination.

FIG. 6 shows the cumulative elution of zotarolimus (percent) compared to the fluocinolone (A109)/zotarolimus (A24) combination.

FIG. 7 shows the cumulative elution of fluocinolone (A109) (μg) in various stent development builds (Groups 1-5).

FIG. 8 shows the cumulative elution of zotarolimus (A24) (μg) in various stent development builds (Groups 1-5).

FIG. 9 shows the daily elution of fluocinolone (A109) (μg) in various stent development builds (Groups 1-5).

FIG. 10 shows the daily elution of zotarolimus (A24) (μg) in various stent development builds (Groups 1-5).

FIG. 11 shows the cumulative elution of fluocinolone (A109) (percent) in various stent development builds (Groups 1-5).

FIG. 12 shows the cumulative elution of zotarolimus (percent) in various stent development builds (Groups 1-5).

### DETAILED DESCRIPTION OF THE INVENTION

The present disclosure generally concerns apparatus and methods related to bioactive agent eluting stents having controlled elution rates. More specifically, the controlled elution rates are achieved by the presence of the bioactive agents themselves.

In one embodiment, the present disclosure relates to a multiple bioactive agent eluting stent comprising a stent framework, at least two bioactive agents on the stent framework, wherein one or more of the at least two bioactive agents affects the elution rate of one or more of the other of the at least two bioactive agents. The elution rate or profile of the bioactive agents can be determined utilizing the methods showing in Examples 1 and 2 below. For example, total cumulative release of applied bioactive agents can be measured to determine total daily release and also release over a period of time greater than one day. The at least two bioactive agents may be applied together onto a stent framework. There may be one coating which contains all of the bioactive agents. Alternatively, the bioactive agents can be applied to the stent framework in separate coatings. Each coating may contain just one bioactive agent or more than one bioactive agent. The coatings may constitute separate layers. There can also be a base layer and cap layer on the stent framework. Base layer is located closest to the stent framework and the cap layer is a layer which is located furthest away from the stent framework. The layers can be uniform or non-uniform, meaning the layers can be in the form of, for example, dots or stripes or other non-uniform patterns.

Alternatively, the multiple bioactive agent eluting stent comprises a stent framework, two bioactive agents on the stent framework, wherein one bioactive agent affects the elution rate of the other bioactive agent.

In another embodiment, the one or more of the at least two bioactive agents increases the elution rate of one or more of the other of the at least two bioactive agents. As shown in present Examples 1 and 2, fluocinolone elution rate is slowed when it is in combination with zotarolimus. This is in contrast to when fluocinolone is present by itself on the tested stents. However, the opposite result is seen with zotarolimus. Zotarolimus elution rate is increased when it is in combination with fluocinolone compared to when zotarolimus is present by itself on the tested stent. Alternatively, the one or more of the at least two bioactive agents decreases the elution rate of one or more of the other of the at least two bioactive agents.

In another embodiment, the effect on elution rate is caused by at least one characteristic of one or more of the at least two bioactive agents. This characteristic may be, for example, hydrophobicity, diffusivity or molecular size. Hydrophobic molecules tend to be antagonistic to water, incapable of dissolving in water. This property is generally characteristic of oils, fats, waxes, and many resins, as well as finely divided powders such as carbon black and magnesium carbonate. Diffusivity of a molecule is shown when there is spontaneous mixing of one substance with another in contact or separated by a permeable membrane or microporous barrier. The rate of diffusion is proportional to the concentration of the substances and increases with temperature. Diffusion occurs most readily in gases, less so in liquids, and least in solids. Size of a molecule can have an effect on its property including its solubility. For example, the solubility of alcohols in water decreases as the molecular size of the alcohol molecule increases. It is the affinity (hydrogen bonding) between the —OH part of the water (H—O—H) molecule and the —OH part of the alcohol (R—OH) that enables them to intermix. When the R— (hydrocarbon tail) part of the alcohol molecule gets larger and larger, the hydrogen bonding effect between the respective solute/solvent —OHs becomes less able to drag the “tail” into solution because the “tail” is compositionally very unlike the water molecule itself. Hydrophobicity, diffusivity and molecule size are just examples of characteristics of the bioactive agents themselves which may affect the elution rate of the other bioactive agent or agents present on the same stent framework.

In another embodiment, the stent framework comprises a metallic base or a polymeric base. Alternatively, the stent framework comprises a material selected from the group consisting of stainless steel, nitinol, tantalum, MP35N alloy, platinum, titanium, a biocompatible alloy, a biocompatible polymer and a combination thereof.

In another embodiment, the multiple bioactive agent eluting stent further comprises one or more polymers on said stent framework.

In another embodiment, the at least two bioactive agents are located in two or more layers on the stent framework. Alternatively, each of the at least two bioactive agents is located in separate layers on the stent framework.

In another embodiment, all of the at least two bioactive agents are in one layer on the stent framework.

In another embodiment, for the multiple bioactive agent eluting stent of the present disclosure, one or more bioactive agents in an inner layer affects the elution rate of one or more bioactive agents in an outer layer.

In another embodiment, at least one of the bioactive agents is selected from the group consisting of an antiproliferative agent, an anti-inflammatory agent, FKBP-12 binding compound, an antisense agent, an antineoplastic agent, an antibiotic, a therapeutic peptide, a gene therapy agent, a therapeutic substance, an organic bioactive agent, a pharmaceutical compound, a recombinant DNA product, a recombinant RNA product, a collagen, a collagenic derivative, a protein, a protein analog, a saccharide, a saccharide derivative, and a combination thereof. Alternatively, the at least one of the bioactive agents is an antiproliferative agent and at least one of the bioactive agents is an anti-inflammatory agent. Alternatively, the antiproliferative agent is selected from the group consisting of zotarolimus, sirolimus, tacrolimus, everolimus, temsirolimus, and the anti-inflammatory agent is selected from the group consisting of glucocorticoids, cyclooxygenase inhibitors, and fluocinolone Alternatively, the antiproliferative agent is zotarolimus (also referred to herein as A24) and the anti-inflammatory agent is fluocinolone (also referred to herein as A109).

In another embodiment, the multiple bioactive agent eluting stent further comprises one or more polymers located on the stent framework. These polymers may be for example, urethanes, polylactides, poly-l-lactic acids, polyglycolic acids, polycaprolactones, polyacrylates, polymethacrylates, polymethylmethacrylates, and combinations and/or copolymers thereof. Alternatively, these polymers may be for example C10, C19, or PVP. Also, the ratio of C10:C19:PVP can be for example, about 30-50:40-70:1-20 or about 38:57:5.

The present disclosure also relates to a method of making a multiple bioactive agent eluting stent comprising the steps of providing a stent framework, applying at least two bioactive agents onto the stent framework, wherein one or more of the at least two bioactive agents affects the elution rate of one or more of the other of the at least two bioactive agents.

In another embodiment, the method of making a bioactive agent eluting stent comprises the steps of providing a stent framework, applying at least two bioactive agents and one or more polymers onto the stent framework, wherein one or more of the at least two bioactive agents affects the elution ratio of one or more of the other of the at least two bioactive agents.

In another embodiment, for the presently disclosed methods, the effect on elution rate is caused by at least one characteristic of one more of the at least two bioactive agents. This characteristic may be, for example, hydrophobicity, diffusivity or molecular size.

In another embodiment, for the presently disclosed methods, each of the at least two bioactive agents is located in a separate layer on the stent framework. In another embodiment, for the presently disclosed methods, all of the at least two bioactive agents are in one layer on the stent framework.

In another embodiment, for the presently disclosed methods, the stent framework comprises a material selected from the group consisting of stainless steel, nitinol, tantalum, MP35N alloy, platinum, titanium, a biocompatible alloy, a biocompatible polymer and a combination thereof.

In another embodiment, for the presently disclosed methods, at least one of the bioactive agents is selected from the group consisting of an antiproliferative agent, an anti-inflammatory agent, FKBP-12 binding compound, an antisense agent, an antineoplastic agent, an antibiotic, a therapeutic peptide, a gene therapy agent, a therapeutic substance, an organic bioactive agent, a pharmaceutical compound, a recombinant DNA product, a recombinant RNA product, a collagen, a collagenic derivative, a protein, a protein analog, a saccharide, a saccharide derivative, and a combination thereof. Alternatively, the at least one of the bioactive agents is an antiproliferative agent and at least one of the bioactive agents is an anti-inflammatory agent. Alternatively, the antiproliferative agent is selected from the group consisting of zotarolimus, sirolimus, tacrolimus, everolimus, temsirolimus, and the anti-inflammatory agent is selected from the group consisting of glucocorticoids, cyclooxygenase inhibitors, and fluocinolone Alternatively, the antiproliferative agent is zotarolimus (also referred to herein as A24) and the anti-inflammatory agent is fluocinolone (also referred to herein as A109).

In another embodiment, for the presently disclosed methods, the bioactive agent eluting stent further comprises one or more polymers located on the stent framework. These polymers may be for example, urethanes, polylactides, poly-l-lactic acids, polyglycolic acids, polycaprolactones, polyacrylates, polymethacrylates, polymethylmethacrylates, and combinations and/or copolymers thereof. Alternatively, these polymers may be for example C10, C19, or PVP. Also, the ratio of C10:C19:PVP can be for example, about 30-50:40-70:1-20 or about 38:57:5.

The following Examples are provided as illustrative embodiments of the present invention. It should be understood that the stents disclosed herein are not limited by the following Examples.

### EXAMPLE 1

12 mm parylene coated stents were used for all builds. Stents were loaded onto 3.0 RX catheters. Formulation shelf life for arms containing fluocinolone (Groups A and B) was limited to 24 hours maximum. Groups C and D had a formulation shelf-life of 72 hrs. Groups A, C and D were dried in vacuum oven at 25° C. and Group B was dried in a class IIB2 laminar flow hood. All groups were dried for a minimum of 12 hours. All groups were EtO (ethylene oxide) sterilized. These bioactive agents were treated as light sensitive. During the spray process, the solution was vortexed prior to filling syringe. No more than 3 ml of solution was added to the syringe during each refill.

TABLE 1
Build Parameters

| | Target | | | | | | | |
|---|---|---|---|---|---|---|---|---|
| | Fluocinolone | | | Dry | Wet | | | |
| | Bioactive | Target | | Coating | Coating | | | |
| | agent Load | Zotarolimus | C10:C19:PVP | Weight | Weight | | | |
| Group | (μg) | Load (μg) | Ratio | (μg) | (μg) | Drying | Sterilization | Solvent |
| A | 0 | 0 | 38:57:5 | 466 +/− 47 | 466 +/− 23 | Vacuum | EtO | Dichloro |
| | | | | | | Oven | | methane |
| B | 0 | 60 | 38:57:5 | 466 +/− 47 | 466 +/− 23 | IIB2 | | (DCM) |
| C | 80 | 0 | 38:57:5 | 466 +/− 47 | 466 +/− 23 | Vacuum | | |
| | | | | | | Oven | | |
| D | 80 | 60 | 38:57:5 | 466 +/− 47 | 466 +/− 23 | Vacuum | | |
| | | | | | | Oven | | |

TABLE 2
Bioactive Agent Content

| | Fluocinolone | Zotarolimus |
|---|---|---|
| Group | Recovery (%) | Recovery (%) |
| A: Polymer Only | 0 | 0 |
| B: Zotarolimus Only | 0 | 104.3 |
| C: Fluocinolone Only | 89.7 | 0 |
| D: Fluocinolone/Zotarolimus | 86.7 | 113.8 |
| Combination | | |
| Fluocinolone/Zotarolimus | 100.7 | 113.6 |
| Combination (Pre-Sterile) | | |

Pre-sterile recovery of fluocinolone in the fluocinolone/zotarolimus arm was found to be 14% higher pre-sterile compared to sterile. zotarolimus recovery was almost identical between sterile and pre-sterile.

Elution results for all three bioactive agent eluting arms would be acceptable for animal implant. Elution of fluocinolone from the fluocinolone only arm was significantly faster than fluocinolone elution from the fluocinolone/zotarolimus arm. The change in elution rate may have been caused by the interaction between the fluocinolone and zotarolimus bioactive agents. Elution for both fluocinolone and zotarolimus was faster, sterile compared to pre-sterile in the fluocinolone/zotarolimus arm.

FIGS. 1-6 are graphs showing the elution rates and profiles of zotarolimus and fluocinolone, obtained using the presently disclosed methods in Example 1.

FIG. 1 shows the cumulative elution of fluocinolone (μg) compared to the fluocinolone/zotarolimus combination. For fluocinolone, the cumulative fluocinolone release over about 10 days was about 62 μg when only fluocinolone was loaded onto the stent. This group is Group C as indicated in Table 1. The initial load for Group C on the stent was 80 μg. For the fluocinolone/zotarolimus combination load on the stent (Group D), the amount of fluocinolone was also 80 μg. In addition to the 80 μg of fluocinolone, 60 μg of zotarolimus was added for Group D. Therefore, the amount of fluocinolone was the same in the combination bioactive agent load (fluocinolone/zotarolimus) and where fluocinolone was present by itself on the stent. For the combination load stent (Group D), fluocinolone total elution or release was less than for Group C where fluocinolone was present by itself. It appears that the presence of zotarolimus affects the elution rate of fluocinolone. In this case, the presence of zotarolimus decreases the elution rate or profile (as measured by total cumulative) release of fluocinolone. This may be due to a characteristic of zotarolimus or fluocinolone, such as hydrophobicity, diffusivity or molecular size.

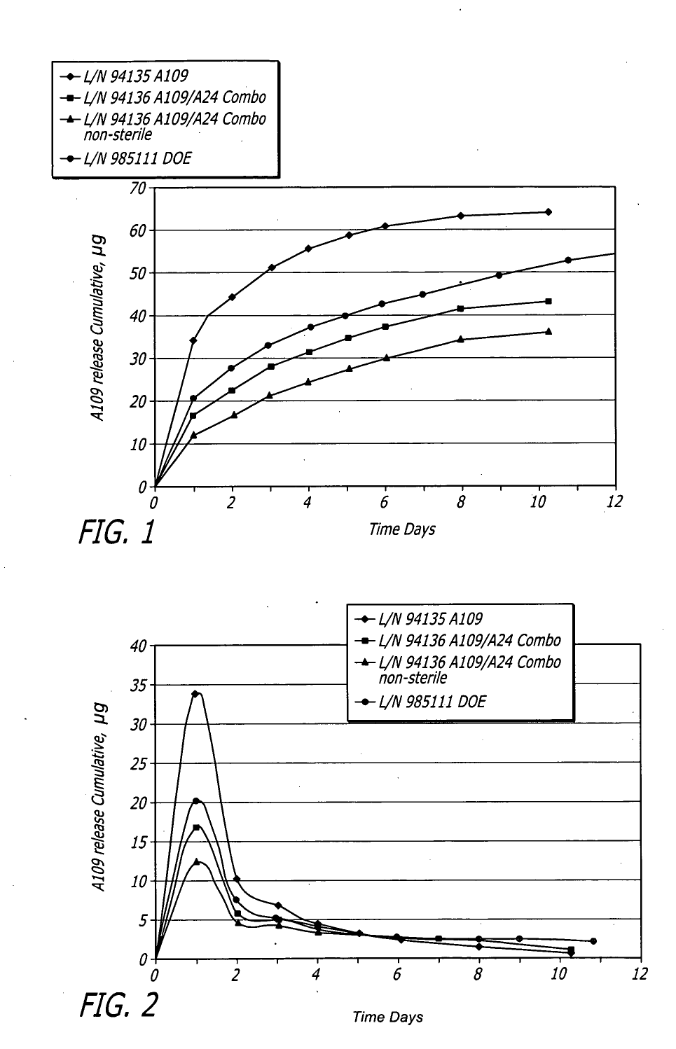

FIG. 2 shows fluocinolone elution rate or profile also. Here, fluocinolone release was measured daily. Representatively on day one, it can be seen that fluocinolone release is greater for Group C than for Group D where a combination of fluocinolone/zotarolimus was loaded onto the tested stent. This result again shows that the presence of zotarolimus affects the elution rate of fluocinolone. This may be due to a characteristic of zotarolimus or fluocinolone, such as hydrophobicity, diffusivity or molecular size.

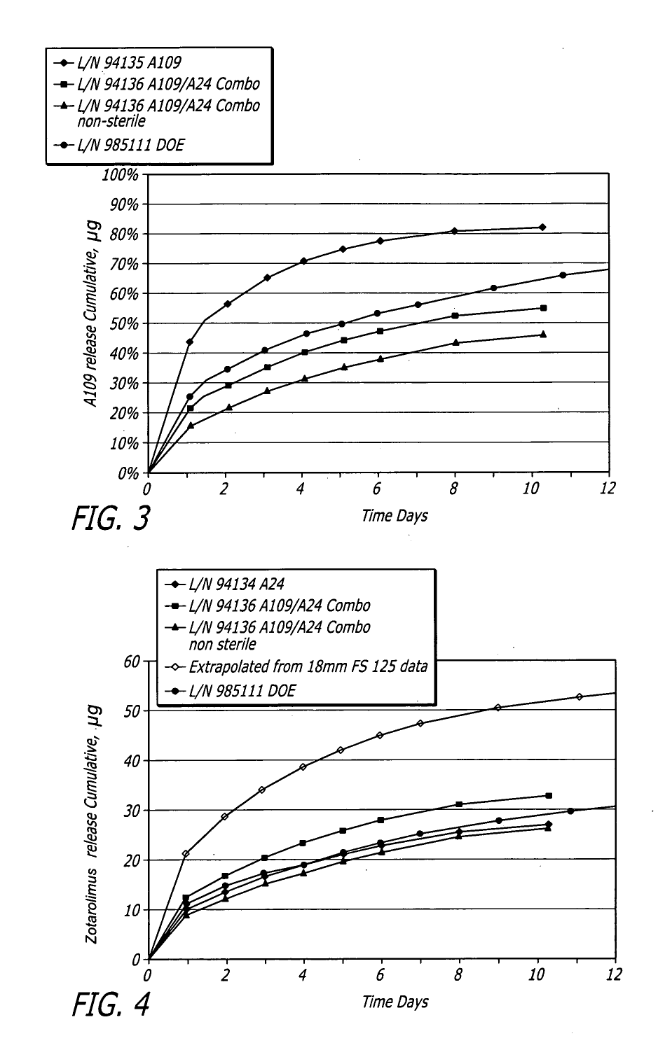

FIG. 3 shows the cumulative elution of fluocinolone (percent) compared to the fluocinolone/zotarolimus combination. This result again shows that the presence of zotarolimus affects the elution rate of fluocinolone. This may be due to a characteristic of zotarolimus or fluocinolone, such as hydrophobicity, diffusivity or molecular size.

FIG. 4 shows the cumulative elution of zotarolimus (μg) compared to the fluocinolone/zotarolimus combination. It can be seen from FIG. 4 that zotarolimus elution rate or profile (as shown here by total cumulative zotarolimus release) is affected by the presence of fluocinolone. However, here the zotarolimus release is greater when in combination with fluocinolone than zotarolimus alone. This is the opposite of the elution rate seen for fluocinolone. It appears that the presence of fluocinolone affects the elution rate of zotarolimus. Here, the presence of fluocinolone increases the elution rate of zotarolimus. This surprising result may be due to a characteristic of zotarolimus or fluocinolone such as hydrophobicity, diffusivity or molecular size.

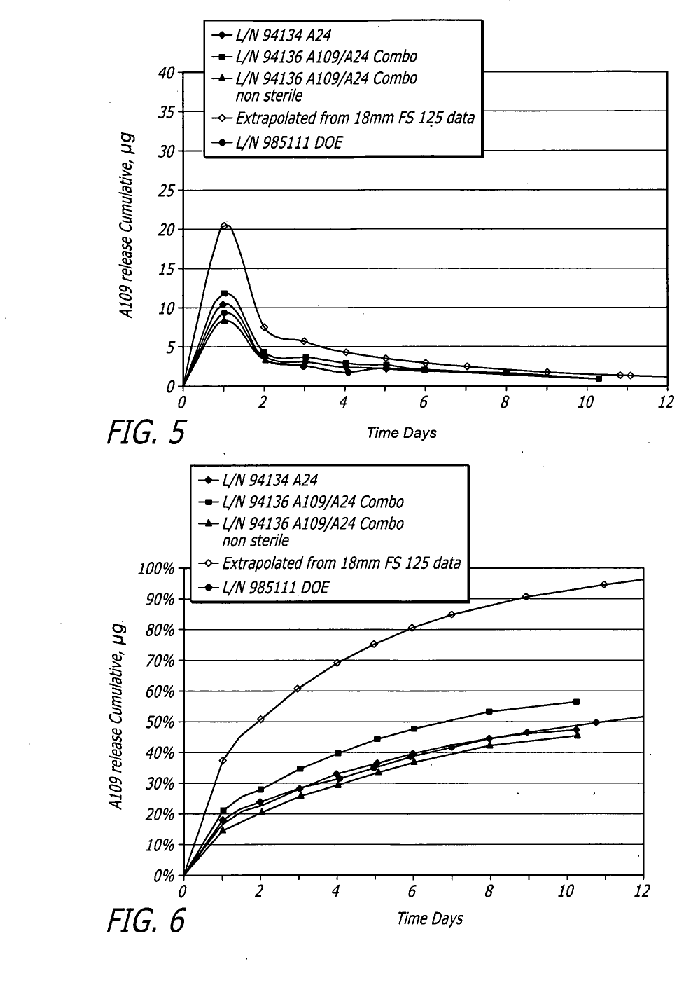

FIG. 5 shows the daily elution of zotarolimus compared to the fluocinolone/zotarolimus combination. Again here, it is seen that zotarolimus elution rate or profile (as shown here by daily measure and record of total cumulative zotarolimus release) is affected by the presence of fluocinolone. Specifically, the presence of fluocinolone increases the elution rate of zotarolimus. This may be due to a characteristic of zotarolimus or fluocinolone such as hydrophobicity, diffusivity or molecular size.

FIG. 6 shows the cumulative elution of zotarolimus (percent) compared to the fluocinolone/zotarolimus combination. This result again shows that the presence of fluocinolone affects the elution rate of zotarolimus. This may be due to a characteristic of zotarolimus or fluocinolone, such as hydrophobicity, diffusivity or molecular size.

### EXAMPLE 2

Medium vessel 3.5×18 mm Driver® stents with a combination of fluocinolone and zotarolimus bioactive agents and C10:C19:PVP polymers (38:57:5) were coated for development of different coating variations.

Stents were coated with a combination of fluocinolone and/or zotarolimus. Group 1 served as a control for 18 mm stent presently tested. Group 2 used the same formulation but with stents pre-crimped to provide preferential outer diameter (OD) coating. Groups 3 to 5 each investigated different layered approaches. Each design maintained the same total amount of each bioactive agent. In Group 3, the bioactive agents were loaded only into the base layer, with a polymer cap coat of equal thickness above. A question to be answered was whether this layering may slow and extend the elution of both bioactive agents, and show whether the cap coat serves as intended or whether the base coat re-solvates and becomes homogeneous throughout. In Groups 4 and 5, the bioactive agents were sprayed in separate layers, bracketing the range of possible dispersions and investigating the relative effect of layer ordering. Layers in these Groups were kept at 30% bioactive agent each to maintain coating durability. If the layering was shown to be effective in altering elution, the information obtained from these Groups might be used to design an optimal coating for a desired bioactive agent release from a stent.

Parylene-coated 3.5×18 mm stents were used for testing, with the different conditions and quantities as listed in Table 3 and Table 4. Units were built per standard coating and device procedures, with formulation shelf life limited to 24 hours maximum and bioactive agents treated as light sensitive. During the spray process, the solution was vortexed prior to filling the syringe. No more than 3 ml of solution was added to the syringe during each refill. Samples were dried for a minimum of 12 hours in a vacuum oven at 25° C., with all testing completed post-sterile.

TABLE 3
Development build table: Base coats.

| | | Bioactive | Wet Coat | Dry Coat | | Sterilization |
|---|---|---|---|---|---|---|
| Group | Polymer | agent Load | Weight | Weight | Solvent | Method |
| 1 | C:10:C19:PVP | 30% | 699 ± 35 μg | 699 ± 70 μg | Methylene | EtO |
| | (38:57:5) | (90 μg | | | Chloride | |
| | | Zotarolimus, | | | Cleaning | |
| | | 120 μg | | | Solvent = | |
| | | Fluocinolone) | | | Methylene | |
| 2 | | 30% | 699 ± 35 μg | 699 ± 70 μg | Chloride | |
| | | (90 μg | | | (for all) | |
| | | Zotarolimus, | | | | |
| | | 120 μg | | | | |
| | | Fluocinolone) | | | | |
| 3 | | 60% | 350 ± 18 μg | 350 ± 35 μg | | |
| | | (90 μg | | | | |
| | | Zotarolimus, | | | | |
| | | 120 μg | | | | |
| | | Fluocinolone) | | | | |
| 4 | | 30% | 399 ± 20 μg | 399 ± 40 μg | | |
| | | (120 μg | | | | |
| | | Fluocinolone) | | | | |
| 5 | | 30% | 300 ± 15 μg | 300 ± 30 μg | | |
| | | (90 μg | | | | |
| | | Zotarolimus) | | | | |

TABLE 4
Development build: Cap coats

| | | Bioactive | Wet Coat | Dry Coat | | Sterilization |
|---|---|---|---|---|---|---|
| Group | Polymer | agent Load | Weight | Weight | Solvent | Method |
| 3 | C:10:C19:PVP | 0% | 699 ± 35 μg | 699 ± 70 μg | Methylene | EtO |
| | (38:57:5) | (0 μg) | | | Chloride | |
| 4 | | 30% | 300 ± 15 μg | 300 ± 30 μg | Cleaning | |
| | | (90 μg | | | Solvent = | |
| | | Zotarolimus, | | | Methylene | |
| | | 120 μg | | | Chloride | |
| | | Fluocinolone) | | | (for all) | |
| 5 | | 60% | 399 ± 20 μg | 399 ± 40 μg | | |
| | | (90 μg | | | | |
| | | Zotarolimus, | | | | |
| | | 120 μg | | | | |
| | | Fluocinolone) | | | | |

TABLE 5
Sample size and test requirement for each combination

| Test | | | | | |
|---|---|---|---|---|---|
| (all post-sterile) | Sample Size | | | | |
| Category | Group 1 | Group 2 | Group 3 | Group 4 | Group 5 |
| Durability/SEM | 2 | 2 | 2 | 2 | 2 |
| Elution | 2 | 2 | 2 | 2 | 2 |
| Bioactive agent | 1 | 2 | 2 | 2 | 2 |
| content | | | | | |
| Retains | 0 | 1 | 0 | 0 | 0 |
| Sum of Units | 5 units | 7 units | 6 units | 6 units | 6 units |

| | |
|---|---|
| TOTAL | 30 units |

Control stents required fewer spray passes (270 to 290) to achieve the target coating weight than stents which had been pre-crimped (340 to 360). Bioactive agent content results were good for all groups, with specific details listed in Table 6 for formulation solutions and Table 7 for stents.

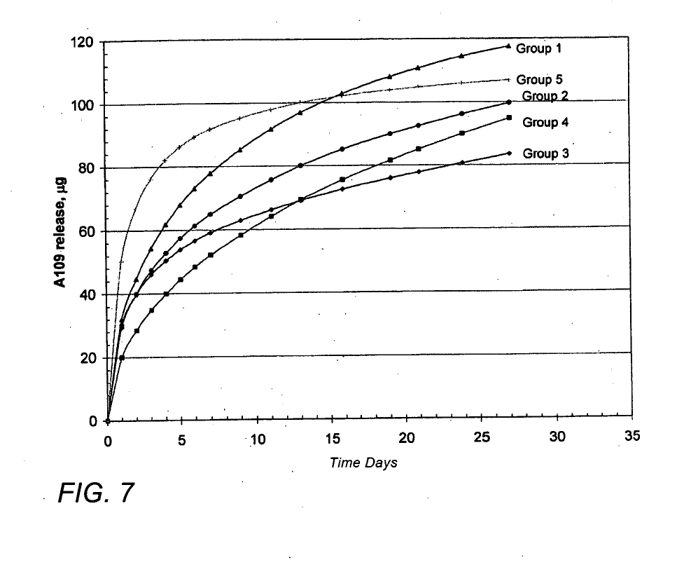

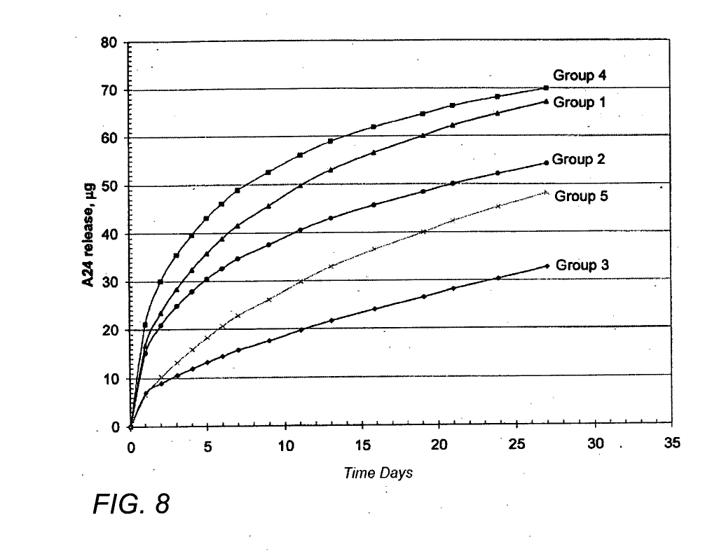

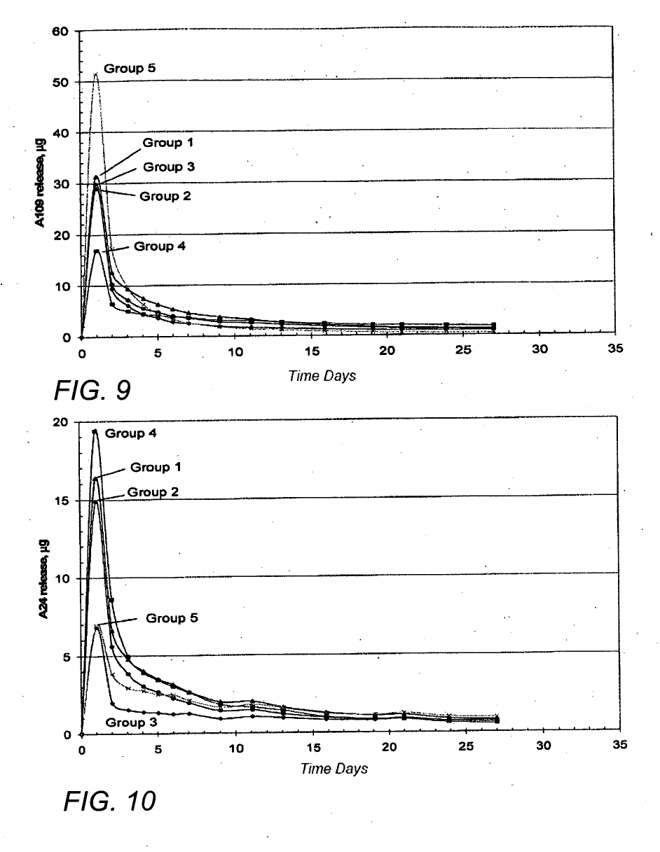

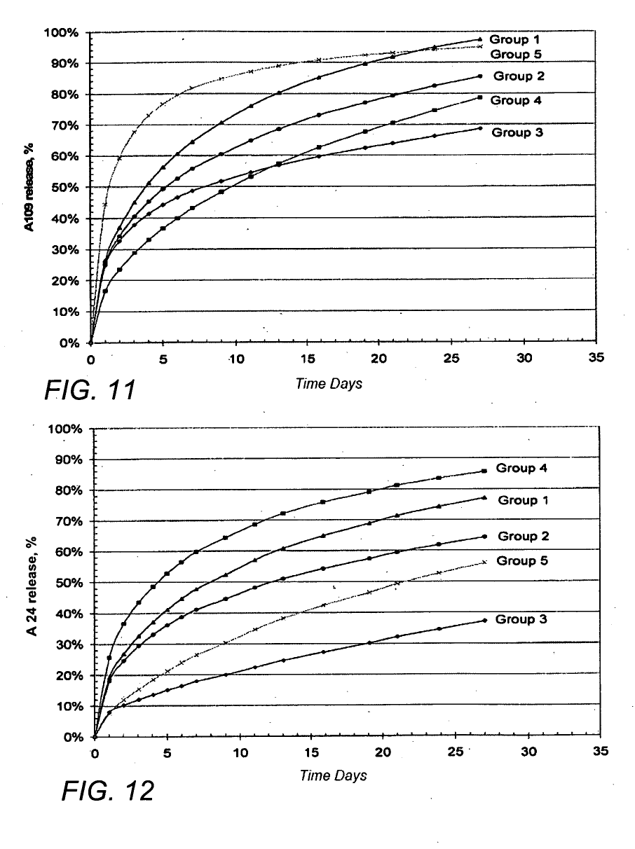

Elution results, including corrections for bioactive agent content, showed a range of curves for fluocinolone release, as detailed in FIG. 7, and for zotarolimus release as detailed in FIG. 8. The pre-crimping slightly slowed down elution, which may be due to the increased OD thickness of these stents, and layers were seen to definitely have an effect. When putting one bioactive agent into the base coat and the other into the cap coat, the base coat bioactive agent's elution is slowed and the cap coat bioactive agent's elution is raised. This effect was greater when zotarolimus was in the base coat and fluocinolone in the cap than the other way around, perhaps related to the different thickness of each bioactive agent layer. The introduction of a large polymer-only cap coat did slow the elution of both bioactive agents, with greater effect on zotarolimus than on fluocinolone.

TABLE 6
Bioactive agent content results for all formulation solutions.

| | Fluocinolone | | | |
|---|---|---|---|---|
| | Bioactive | Zotarolimus | Actual | Actual |
| | agent | Bioactive agent | Fluocinolone | Zotarolimus |
| Solution | Load, % | Load, % | recovery, % | recovery, % |
| Group 1, 2 | 17.0% | 13% | 103.6% | 101.0% |
| Group 3 base | 34.0% | 26.0% | 97.4% | 100.7% |
| Group 4 base | 30.0% | 0.0% | 99.2% | NA |
| Group 5 base | 0.0% | 30.0% | NA | 99.6% |
| Group 3 base | 0.0% | 0.0% | NA | NA |
| Group 4 base | 30.0% | 0.0% | 94.9% | NA |
| Group 5 base | 0.0% | 30.0% | NA | 100.4% |

TABLE 7
Bioactive agent content results for all stent groups.

| | Fluocinolone | Zotarolimus | Average | Average |
|---|---|---|---|---|
| | Bioactive agent | Bioactive agent | Fluocinolone | Zotarolimus |
| Group | Load, % | Load, % | recovery, % | recovery, % |
| 1 | 17% | 13% | 104.4% | 96.4% |
| 2 | 17% | 13% | 105.8% | 97.7% |
| 3 | 34% | 26% | 99.9% | 97.3% |
| 4 | 30% | 30% | 100.6% | 98.5% |
| 5 | 30% | 30% | 96.4% | 98.0% |

This build showed good results for all groups, and indicated a clear difference in bioactive agent elution based on whether the coating is applied as a single cocktail or in segregated layers.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A multiple bioactive agent eluting stent comprising:
a stent framework;
at least two bioactive agents on said stent framework;
wherein one or more of the said at least two bioactive agents affects the elution rate of one or more of the other of said at least two bioactive agents.

2. The multiple bioactive agent eluting stent of claim 1, comprising:
a stent framework;
two bioactive agents on said stent framework;
wherein one bioactive agent affects the elution rate of the other bioactive agent.

3. The multiple bioactive agent eluting stent of claim 1, wherein one or more of the said at least two bioactive agents increases the elution rate of one or more of the other of said at least two bioactive agents.

4. The multiple bioactive agent eluting stent of claim 1, wherein one or more of the said at least two bioactive agents decreases the elution rate of one or more of the other of said at least two bioactive agents.

5. The multiple bioactive agent eluting stent of claim 1, wherein said effect on elution rate is caused by at least one characteristic of one or more of said at least two bioactive agents.

6. The multiple bioactive agent eluting stent of claim 5, wherein said characteristic is hydrophobicity, diffusivity, or molecular size.

7. The multiple bioactive agent eluting stent of claim 1, wherein said stent framework comprises a metallic base or a polymeric base.

8. The multiple bioactive agent eluting stent of claim 1, wherein the stent framework comprises a material selected from the group consisting of stainless steel, nitinol, tantalum, MP35N alloy, platinum, titanium, a biocompatible alloy, a biocompatible polymer, and a combination thereof.

9. The multiple bioactive agent eluting stent of claim 1, further comprising one or more polymers on said stent framework.

10. The multiple bioactive agent eluting stent of claim 1, wherein said at least two bioactive agents are located in two or more layers on said stent framework

11. The multiple bioactive agent eluting stent of claim 1, wherein each of said at least two bioactive agents is located in separate layers on said stent framework.

12. The multiple bioactive agent eluting stent of claim 1, wherein all of said at least two bioactive agents are in one layer on said stent framework.

13. The multiple bioactive agent eluting stent of claim 10, wherein one or more bioactive agents in an inner layer affects the elution rate of one or more bioactive agents in an outer layer.

14. The multiple bioactive agent eluting stent of claim 1, wherein at least one of said bioactive agents is selected from the group consisting of an antiproliferative agent, an anti-inflammatory agent, FKBP-12 binding compound, an antisense agent, an antineoplastic agent, an antibiotic, a therapeutic peptide, a gene therapy agent, a therapeutic substance, an organic bioactive agent, a pharmaceutical compound, a recombinant DNA product, a recombinant RNA product, a collagen, a collagenic derivative, a protein, a protein analog, a saccharide, a saccharide derivative, and a combination thereof.

15. The multiple bioactive agent eluting stent of claim 1, wherein at least one of said bioactive agents is an antiproliferative agent and at least one of said bioactive agents is an anti-inflammatory agent.

16. The multiple bioactive agent eluting stent of claim 15, wherein said antiproliferative agent is selected from the group consisting of zotarolimus, sirolimus, tacrolimus, everolimus, temsirolimus, and said anti-inflammatory agent is selected from the group consisting of glucocorticoids, cyclooxygenase inhibitors, and fluocinolone.

17. The multiple bioactive agent eluting stent of claim 15, wherein said antiproliferative agent is zotarolimus and said anti-inflammatory agent is fluocinolone.

18. The multiple bioactive agent eluting stent of claim 9, wherein said one or more polymers is/are selected from the group consisting of urethanes, polylactides, poly-l-lactic acids, polyglycolic acids, polycaprolactones, polyacrylates, polymethacrylates, polymethylmethacrylates, and combinations and/or copolymers thereof.

19. The multiple bioactive agent eluting stent of claim 9, wherein said polymers are C10, C19, and PVP.

20. The multiple bioactive agent eluting stent of claim 19, wherein the ratio of C10, C19, and PVP is about 30-50:40-70:1-20.

21. The multiple bioactive agent eluting stent of claim 19, wherein the ratio of C10:C19:PVP is about 38:57:5.

22. A method of making a bioactive agent eluting stent comprising the steps of:
providing a stent framework;
applying at least two bioactive agents onto said stent framework;
wherein one or more of the said at least two bioactive agents affects the elution rate of one or more of the other of said at least two bioactive agents.

23. The method of claim 22, comprising the steps of
providing a stent framework;
applying at least two bioactive agents and one or more polymers onto said stent framework;
wherein one or more of said at least two bioactive agents affects the elution rate of one or more of the other of said at least two bioactive agents.

24. The method of claim 22, wherein said effect on elution rate is caused by at least one characteristic of one or more of said at least two bioactive agents.

25. The method of claim 22, wherein said characteristic is hybrophobicity, diffusivity, or molecular size.

26. The method of claim 22, wherein each of said at least two bioactive agents is located in a separate layer on said stent framework.

27. The method of claim 22, wherein all of said at least two bioactive agents are in one layer on said stent framework.

28. The method of claim 22, wherein the stent framework comprises a material selected from the group consisting of stainless steel, nitinol, tantalum, MP35N alloy, platinum, titanium, a biocompatible alloy, a biocompatible polymer, and a combination thereof.

29. The method of claim 22, wherein at least one of said bioactive agents is selected from the group consisting of an antiproliferative agent, an anti-inflammatory agent, FKBP-12 binding compound, an antisense agent, an antineoplastic agent, an antibiotic, a therapeutic peptide, a gene therapy agent, a therapeutic substance, an organic bioactive agent, a pharmaceutical compound, a recombinant DNA product, a recombinant RNA product, a collagen, a collagenic derivative, a protein, a protein analog, a saccharide, a saccharide derivative, and a combination thereof.

30. The method of claim 22, wherein at least one of said bioactive agents is an antiproliferative agent and at least one of said bioactive agents is an anti-inflammatory agent.

31. The method of claim 30, wherein said antiproliferative agent is selected from the group consisting of zotarolimus, sirolimus, tacrolimus, everolimus, temsirolimus, and said anti-inflammatory agent is selected from the group consisting of glucocorticoids, cyclooxygenase inhibitors, and fluocinolone.

32. The method of claim 31, wherein said antiproliferative agent is zotarolimus and said anti-inflammatory agent is fluocinolone.

33. The method of claim 23, wherein said one or more polymers is/are selected from the group consisting of urethanes, polylactides, poly-l-lactic acids, polyglycolic acids, polycaprolactones, polyacrylates, polymethacrylates, polymethylmethacrylates, and combinations and/or copolymers thereof.

34. The method of claim 23, wherein said polymers are C10, C19, and PVP.

35. The method of claim 34, wherein the ratio of C10:C19: PVP is about 30-50:40-70:1-20.

36. The method of claim 35, wherein the ratio of C10:C19: PVP is about 38:57:5.
